(19) United States
(12) Patent Application Publication
(10) Pub. No.: US 2017/0298006 A1
(43) Pub. Date: Oct. 19, 2017

(54) SUBSTITUTED 1,2-DIHYDROQUINOLINES WITH LOW CONTAMINANT LEVELS AND PROCESSES FOR PREPARING

(71) Applicant: Novus International Inc., St. Charles, MO (US)

(72) Inventors: Graciela B. Arhancet, St. Charles, MO (US); Matthew W. Mahoney, St. Charles, MO (US); Scott A. Long, St. Charles, MO (US); Xiaojun Wang, St. Charles, MO (US); Andrei D. Stefanescu, St. Charles, MO (US); Jason W. Schultz, St. Charles, MO (US)

(21) Appl. No.: 15/488,695

(22) Filed: Apr. 17, 2017

Related U.S. Application Data
(60) Provisional application No. 62/324,037, filed on Apr. 18, 2016.

(51) Int. Cl. C07C 217/84; C07D 215/04; C07C 213/10

(52) U.S. Cl. CPC C07C 217/84; C07C 213/10; C07D 215/04

(57) ABSTRACT

Processes for preparing compositions comprising substituted 1,2-dihydroquinolines and having low or undetectable levels of substituted anilines, wherein the processes comprise removing the substituted anilines from feed streams comprising the substituted 1,2-dihydroquinolines and substituted anilines.

### CROSS-REFERENCE TO RELATED APPLICATIONS

This application claims the priority of U.S. Provisional Application Ser. No. 62/324,037, filed Apr. 18, 2016, the disclosure of which is hereby incorporated by reference in its entirety.

### FIELD

The present disclosure generally relates to compositions comprising substituted 1,2-dihydroquinolines and low levels of substituted anilines carried over from synthesis of the substituted 1,2-dihydroquinolines, and processes for removing the contaminating substituted anilines.

### BACKGROUND

Some substituted 1,2-dihydroquinolines have utility as antioxidants. For example, 6-ethoxy-2,2,4-trimethyl-1,2-dihydroquinoline (whose common name is ethoxyquin) is widely used in the animal feed industry to prevent oxidation of lipids or fats in feeds and in the pigment industry to prevent color loss due to oxidation of the natural carotenoid pigments. Substituted 1,2-dihydroquinolines can be synthesized by condensation of a substituted aniline and a compound containing a carbonyl group. For example, ethoxyquin is routinely prepared by condensing para-phenetidine with acetone in the presence of an acid catalyst. Commercial samples of ethoxyquin can contain up to 30,000 ppm of residual p-phenetidine, but recent improvements in commercial processing have resulted in samples with p-phenetidine levels as low as about 100-200 ppm. While ethoxyquin is considered non-toxic, p-phenetidine is a possible mutagen. Thus, there is a need for methods to remove substituted anilines, such as p-phenetidine, from preparations comprising substituted 1,2-dihydroquinolines.

### SUMMARY

Among the various aspects of the present disclosure encompasses a composition comprising a substituted 1,2-dihydroquinoline of Formula (I) and less than about 40 ppm of a substituted aniline of Formula (II), wherein the substituted 1,2-dihydroquinoline of Formula (I) is produced in a batch of at least one metric ton by reacting the substituted aniline of Formula (II) with a compound comprising a carbonyl group. The compounds of Formulas (I) and (II) having the following structures:

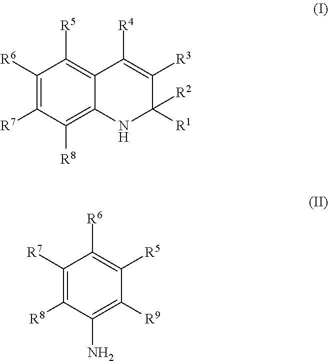

wherein:
R¹, R², R³ and R⁴ are independently hydrogen, C₁-C₆ alkyl, or C₁-C₆ substituted alkyl;
R⁵, R⁷, and R⁹ are independently hydrogen, C₁-C₆ alkyl, or C₁-C₆ substituted alkyl; and
R⁶ and R⁸ are independently hydrogen, C₁-C₆ alkoxy, or C₁-C₆ substituted alkoxy group, provided that one is other than hydrogen.

Another aspect of the present disclosure encompasses a process for removing a compound of Formula (II) from a feed stream comprising a compound of Formula (I) and the compound of Formula (II). The process comprises (a) contacting the feed stream comprising the compounds of Formula (I) and Formula (II) with an aqueous solution having a pH from about 2 to 4 to form an aqueous phase comprising the compound of Formula (II) and an organic phase comprising the compound of Formula (I); (b) separating the phases; and (c) isolating the compound of Formula (I) from the organic phase to yield a purified preparation of the compound of Formula (I), wherein the purified preparation of the compound of Formula (I) contains less than about 70 ppm of the compound of Formula (II). The compounds of Formulas (I) and (II) have the following structures:

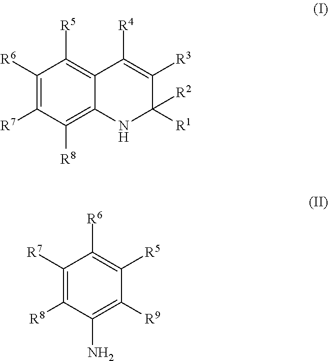

wherein:
R¹, R², R³ and R⁴ are independently hydrogen, C₁-C₆ alkyl, or C₁-C₆ substituted alkyl;
R⁵, R⁷, and R⁹ are independently hydrogen, C₁-C₆ alkyl, or C₁-C₆ substituted alkyl; and
R⁶ and R⁸ are independently hydrogen, C₁-C₆ alkoxy, or C₁-C₆ substituted alkoxy group, provided that one is other than hydrogen.

A further aspect of the present disclosure provides another process for removing a compound of Formula (II) from a feed stream comprising a compound of Formula (I) and the compound of Formula (II). The process comprises contacting the feed stream comprising the compounds of Formula (I) and Formula (II) with a resin to selectively remove the compound of Formula (II) and yield a purified preparation of the compound of Formula (I), wherein the purified preparation of the compound of Formula (I) contains less than about 70 ppm of the compound of Formula (II). The compounds of Formulas (I) and (II) have the following structures:

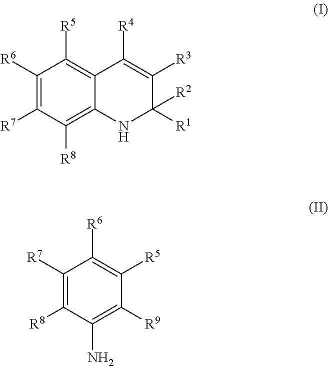

wherein:
R¹, R², R³ and R⁴ are independently hydrogen, C₁-C₆ alkyl, or C₁-C₆ substituted alkyl;
R⁵, R⁷, and R⁹ are independently hydrogen, C₁-C₆ alkyl, or C₁-C₆ substituted alkyl; and
R⁶ and R⁸ are independently hydrogen, C₁-C₆ alkoxy, or C₁-C₆ substituted alkoxy group, provided that one is other than hydrogen.

Other features and iterations of the disclosure are described in more detail below.

### BRIEF DESCRIPTION OF THE DRAWINGS

FIG. 1 presents p-phenetidine (PPN) levels in ethoxyquin (EQ) after extraction with aqueous HCl solutions of increasing pH. The levels of PPN in EQ are plotted as a function of the level of pH of the aqueous extraction solution.

FIG. 2 illustrates recovery of EQ after extraction with aqueous HCl solutions of increasing pH. The percent recovery of EQ is plotted as a function of the level of pH of the aqueous extraction solution.

### DETAILED DESCRIPTION

The present disclosure provides compositions comprising substituted 1,2-dihydroquinolines and substituted anilines, wherein the levels of the substituted anilines generally are less than about 70 ppm. Also provided are processes for removing substituted anilines from preparations of the substituted 1,2-dihydroquinolines. The processes are designed to exploit functional differences between the two compounds (e.g., different water solubility, different polarity, different reactive groups, and so forth) such that the two compounds can be separated.

### (I) Compositions

One aspect of the present disclosure provides compositions comprising substituted 1,2-dihydroquinoline and less than about 70 ppm of substituted aniline, wherein the substituted 1,2-dihydroquinoline is produced at an industrial scale by reacting the substituted aniline with a compound comprising a carbonyl group.

(a) Substituted 1,2-Dihydroquinoline

The substituted 1,2-dihydroquinoline is a compound of Formula (I):

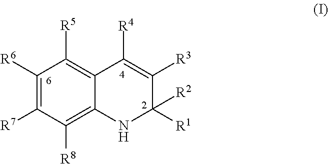

wherein:
R¹, R², R³ and R⁴ are independently hydrogen, C₁-C₆ alkyl, or C₁-C₆ substituted alkyl;
R⁵ and R⁷ are independently hydrogen, C₁-C₆ alkyl, or C₁-C₆ substituted alkyl; and
R⁶ and R⁸ are independently hydrogen, C₁-C₆ alkoxy, or C₁-C₆ substituted alkoxy group, provided that one is other than hydrogen.

In various embodiments, R¹, R², R³ and R⁴ are independently hydrogen, methyl, ethyl, propyl, isopropyl, butyl, sec-butyl, isobutyl, tert-butyl, pentyl, neopentyl, isopentyl, hexyl, or isohexyl. In specific embodiments, R¹, R², R³ and R⁴ are independently hydrogen or methyl. In one embodiment, each of R¹ and R² is methyl, and each of R³ and R⁴ is hydrogen. In another embodiment, each of R¹, R², and R⁴ is methyl, and R³ is hydrogen.

In some embodiments, R⁵ and R⁷ are independently hydrogen, methyl, ethyl, propyl, isopropyl, butyl, sec-butyl, isobutyl, tert-butyl, pentyl, neopentyl, isopentyl, hexyl, or isohexyl. In specific embodiments, R⁵ and R⁷ are hydrogen.

In certain embodiments, R⁶ and R⁸ are independently hydrogen, methoxy, ethoxy, propoxy, or butoxy. In some embodiments, R⁶ is hydrogen and R⁸ is ethoxy. In exemplary embodiments, R⁸ is hydrogen and R⁶ is ethoxy.

In specific embodiments, each of R¹, R², and R⁴ is methyl, each of R³, R⁵, R⁷, and R⁸ is hydrogen, and R⁶ is ethoxy. Stated another way, the substituted 1,2-dihydroquinoline is 6-ethoxy-2,2,4-trimethyl-1,2-dihydroquinoline (also known as ethoxyquin), which is diagrammed below.

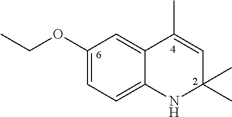 [SMILES: CCOc1ccc2c(c1)C(C)=CC(C)(C)N2]

The amount of the compound of Formula (I) present in the compositions disclosed herein may vary. In general, the composition contains at least about 80% of the compound of Formula (I). In some embodiments, the concentration of the compound of Formula (I) in the composition may be at least about 85% by weight. In other embodiments, the concentration of the compound of Formula (I) in the composition may be at least about 90% by weight. In various embodiments, the concentration of the compound of Formula (I) in the composition may be at least about 90%, at least about 91%, at least about 92%, at least about 93%, at least about 94%, at least about 95%, at least about 96%, at least about 97%, at least about 98%, or at least about 99%.

The compositions disclosed herein may contain polymers and/or breakdown products of the compound of Formula (I) (e.g., dimers, imines, N-oxides, and the like). For example, the composition may contain less than about 20%, less than about 15%, less than about 10%, less than about 5%, less than about 4%, less than about 3%, less than about 2%, or less than about 1% of polymers and/or breakdown products of the compound of Formula (I).

(b) Substituted Aniline

The substituted aniline is a compound of Formula (II):

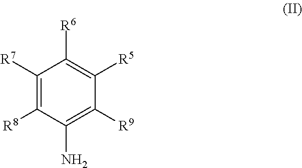

wherein:
R⁵, R⁷, and R⁹ are independently hydrogen, C₁-C₆ alkyl, or C₁-C₆ substituted alkyl; and
R⁶ and R⁸ are independently hydrogen, C₁-C₆ alkoxy, or C₁-C₆ substituted alkoxy group, provided that one is other than hydrogen.

In some embodiments, R⁵, R⁷, and R⁹ are independently hydrogen, methyl, ethyl, propyl, isopropyl, butyl, sec-butyl, isobutyl, tert-butyl, pentyl, neopentyl, isopentyl, hexyl, or isohexyl. In specific embodiments, R⁵, R⁷, and R⁹ are hydrogen.

In certain embodiments, R⁶ and R⁸ are independently hydrogen, methoxy, ethoxy, propoxy, or butoxy. In some embodiments, R⁶ is hydrogen and R⁸ is ethoxy. In exemplary embodiments, R⁸ is hydrogen and R⁶ is ethoxy.

In specific embodiments, R⁵, R⁷, R⁸, and R⁹ are hydrogen and R⁶ is ethoxy. In other words, the substituted aniline is p-phenetidine (also known as 4-ethoxyaniline), which is shown below.

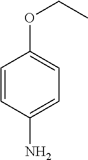

The amount of the compound of Formula (II) present in the compositions disclosed herein is generally less than about 70 ppm. In some embodiments, the concentration of the compound of Formula (II) may be less than about 65 ppm, less than about 60 ppm, less than about 55 ppm, less than about 50 ppm, less than about 45 ppm, less than about 40 ppm, less than about 39 ppm, less than about 37, less than about 35 ppm, less than about 33 ppm, less than about 30 ppm, less than about 25 ppm, less than about 20 ppm, less than about 15 ppm, less than about 10 ppm, less than about 5 ppm, less than about 1 ppm, or below the limit of detection.

(c) Production Means

The compositions comprising the substituted 1,2-dihydroquinoline of Formula (I) disclosed herein are produced at an industrial scale by reacting the substituted aniline of Formula (II) with a compound comprising a carbonyl. It is generally recognized that industrial scale refers to production batches of at least about 100 kg. In various embodiments, each production batch of the substituted 1,2-dihydroquinoline of Formula (I) is at least about 200 kg, at least about 500 kg, at least about 1,000 kg (1 metric ton), at least about 2 metric tons, at least about 5 metric tons, at least about 10 metric tons, at least about 20 metric tons, at least about 50 metric tons, at least about 100 metric tons, or more than about 100 metric tons. The industrially-produced compositions disclosed herein, therefore, contain residual levels of the starting substituted aniline compound of Formula (II). In specific embodiments, the compositions comprise 6-ethoxy-2,2,4-trimethyl-1,2-dihydroquinoline and less than about 70 ppm of p-phenetidine.

(d) Means for Detecting the Substituted Aniline

The level of the substituted aniline of Formula (II) in the compositions disclosed herein may be measured using a variety of analytical methods. In some embodiments, the analytical method may a gas chromatography method. The gas chromatograph analysis may be coupled with a variety of detection modes. Suitable detection modes include flame ionization and mass spectrometry.

In some embodiments, the level of the substituted aniline may be measured using a gas chromatography-flame ionization detection (GC-FID) method. This method is based on the FCC (Food Chemicals Codex, version 6) method, with addition of a calibration curve and increased sample concentration. Adjustments were also made to the instrumental method to increase sensitivity. The equipment needed includes a GC equipped with helium or nitrogen carrier gas, capillary injector with split insert, electronic flow control, flame ionization detector and a 10V output, appropriate data collection system, and a Restek Rtx-5MS capillary column (or equivalent) (30 m×0.25 mm×0.25 μm).

In other embodiments, the level of the substituted aniline may be measured using a gas chromatography-mass spectrometry (GC-MS) method. This method is based on the GC-FID method described above except the GC is linked to MS detector and the operating parameters are optimized.

(e) Properties of the Compositions

The compositions disclosed herein are generally liquid compositions. These compositions are substantially devoid of water and/or solvent. The term “substantially devoid” means that no water and/or solvent can be detected in the composition using standard analytical methods used to detect water and/or organic solvents. In some embodiments, the compositions contain less than about 2.0%, less than about 1.5%, less than 1.0%, less than 0.5%, or less than 0.1% by weight of water and/or solvent.

The compositions disclosed herein are substantially stable, i.e., the concentration of the substituted aniline of Formula (II) does not significantly change over time. In some embodiments, the concentration of the substituted aniline of Formula (II) increases less than about 10% over time. In other embodiments, the concentration of the substituted aniline of Formula (II) increases less than about 8%, less than about 6%, less than about 4%, less than about 2%, or less than about 1% over time. In some embodiments, the compositions disclosed herein are substantially stable for at least one month, at least three months, at least six months, at least one year, or for longer than one year when stored at room temperature (i.e., about 20-23° C.) under standard conditions (e.g., in closed containers with no exposure to light).

The compositions disclosed herein are substantially devoid of imine derivatives of the substituted aniline of Formula (II), which means that imine derivatives are undetectable using standard analytical methods. It is hypothesized that the imine derivatives can be formed during the synthesis reaction by condensation of the substituted aniline of Formula (II) and multiple molecules of the carbonyl-containing compound, and that the imine derivatives can revert back to the substituted aniline of Formula (II) under acidic conditions. Accordingly, the levels of the substituted aniline of Formula (II) in the compositions disclosed herein do not increase in the presence of acid.

(f) Exemplary Compositions

In some embodiments, the substituted 1,2-dihydroquinoline of Formula (I) is 6-ethoxy-2,2,4-trimethyl-1,2-dihydroquinoline and the substituted aniline of Formula (II) is p-phenetidine. The composition comprises less than about 70 ppm of p-phenetidine. In some embodiments, the concentration of p-phenetidine in the composition is less than about 40 ppm. In other embodiments, the concentration of p-phenetidine in the composition is less than about 20 ppm. In additional embodiments, the concentration of p-phenetidine in the composition is less than about 10 ppm. In still other embodiments, the concentration of p-phenetidine is below the limit of detection.

### (II) Purification Processes

Another aspect of the present disclosure encompasses processes for removing substituted anilines of Formula (II) from industrial feed streams or solutions comprising substituted 1,2-dihydroquinolines of Formula (I) and substituted anilines of Formula (II), thereby providing purified preparations of the substituted 1,2-dihydroquinolines of Formula (I) having low or undetectable levels of the substituted anilines of Formula (II). Several processes are disclosed below. A single process may be used to remove the substituted aniline of Formula (II) or one or more processes may be used sequentially (in any order) to remove the substituted aniline of Formula (II) from a feed stream or solution comprising the compounds of Formula (I) and Formula (II). The resultant compositions are detailed above in section (I).

In specific embodiments, the substituted 1,2-dihydroquinoline of Formula (I) is 6-ethoxy-2,2,4-trimethyl-1,2-dihydroquinoline and the substituted aniline of Formula (II) is p-phenetidine.

(a) Extracting with Acidic Aqueous Solution

One process for removing the substituted aniline of Formula (II) from a feed stream or solution comprising the substituted 1,2-dihydroquinoline of Formula (I) and the substituted aniline of Formula (II) comprises a) contacting the feed stream/solution with an acidic aqueous solution to form an aqueous phase comprising the substituted aniline of Formula (II) and an organic phase comprising the compound of Formula (I); (b) separating the phases; and (c) isolating the compound of Formula (I) from the organic phase to yield a purified preparation of the substituted 1,2-dihydroquinoline of Formula (I).

(i) Acidic Aqueous Solution

The acidic aqueous solution that is used to remove the substituted aniline of Formula (II) from the feed stream or solution comprising the compounds of Formula (I) and Formula (II) may comprise an inorganic acid or an organic acid diluted in water. Non-limiting examples of suitable inorganic acids include hydrochloric acid, nitric acid, phosphoric acid, sulfuric acid, boric acid, hydrofluoric acid, hydrobromic acid, hydroiodic acid, perchloric acid, or combinations thereof. Suitable organic acids include, without limit, carboxylic acids such as formic acid, acetic acid, propionic acid, citric acid, lactic acid, malic acid, benzoic acid, and so forth, and sulfonic acids such p-toluenesulfonic acid, trifluoromethanesulfonic acid, and the like. In specific embodiments, the acidic aqueous solution may comprise hydrochloric acid and water. In other embodiments, the acidic aqueous solution may comprise formic acid.

The concentration of acid in the acidic aqueous solution can and will vary depending upon the identity of the acid. In embodiments in which the acid is hydrochloric acid, the concentration of acid may be about 0.1 M, about 0.01 M. about 0.001 M, or about 0.0001 M. In embodiments in which the acid is formic acid, the concentration of acid may be about 0.9 M, about 1.8 M, about 3.6 M, about 7.2 M, or about 14.4 M.

In general, the acidic aqueous solution has a pH value that ranges from about pH 1.5 to about pH 4.5. In some embodiments, the pH of the acidic aqueous solution ranges from about pH 1.8 to about pH 4.2, from about 1.9 to about 4.1, or from about pH 2 to about pH 4. In other embodiments, the pH of the acidic aqueous solution ranges from about pH 1.9 to about pH 3. In still other embodiments, the pH of the acidic aqueous solution ranges from about pH 3 to about pH 4.1.

(ii) Optional Solvent

The feed stream or solution comprising the compounds of Formula (I) and Formula (II) may be used as is (i.e., neat) or it may be mixed with a suitable solvent. In general, the substituted 1,2-dihydroquinoline of Formula (I) is water insoluble, but is soluble in nonpolar solvents. Non-limiting examples of suitable nonpolar solvents include benzene, n-butanol, butyl acetate, carbon tetrachloride, chloroform, cyclohexane, 1,2-dichloroethane, dichloromethane, ethyl acetate, di-ethyl ether, heptane, hexane, methyl-t-butyl ether, methyl ethyl ketone, pentane, di-iso-propyl ether, toluene, trichloroethylene, or combinations thereof. In specific embodiments, the solvent may be toluene, heptane, or hexanes.

In embodiments in which the solvent is used, the ratio (v/w) of the solvent to the substituted 1,2-dihydroquinoline of Formula (I) can and will vary depending, for example, on the identity of the substituents on the substituted 1,2-dihydroquinoline of Formula (I) and the identity of the solvent. In various embodiments, the substituted 1,2-dihydroquinoline of Formula (I) may be dissolved in an appropriate volume of the solvent to prepare a solution comprising about 1% to about 95% (by weight) of the substituted 1,2-dihydroquinoline of Formula (I). In various embodiments, the solution may contain from about 1% to about 10%, from about 10% to about 30%, from about 30% to about 60%, or from about 60% to about 95% of the substituted 1,2-dihydroquinoline of Formula (I).

(iii) Extracting and Separating the Phases

The process comprises contacting the feed stream or solution comprising the substituted 1,2-dihydroquinoline of Formula (I) and the substituted aniline of Formula (II) with the acidic aqueous solution. The ratio (v/v) of the feed stream/solution comprising said compounds to the acidic aqueous solution may vary. In some embodiments, the v/v ratio of the feed stream/solution to the acidic aqueous solution may range from about 1:0.01 to about 1:10. In various embodiments, the v/v ratio of the feed stream/solution to the acidic aqueous solution may range from about 1:0.01 to about 1:0.1, from about 1:0.1 to about 1:0.3, from about 1:0.3 to about 1:1, from about 1:1 to about 1:3, or from about 1:3 to about 1:10.

Contact between the feed stream or solution comprising the compounds of Formula (I) and Formula (II) and the acidic aqueous solution may occur at a temperature ranging from about 4° C. to about 60° C. In some embodiments, the temperature of the contacting step may range from about 0° C. to about 20° C., from about 20° C. to about 30° C., from about 30° C. to about 40° C., or from about 40° C. to about 60° C. In specific embodiments, the contacting may be performed at about room temperature.

Contacting the feed stream or solution comprising the substituted 1,2-dihydroquinoline of Formula (I) and the substituted aniline of Formula (II) with the acidic aqueous solution forms a two phase system. The two phase system may be mixed by inversion, rotation, stirring, shaking, or other means known in the art. After the mixing, the system is allowed to separate into two phases, i.e., an aqueous phase comprising the substituted aniline of Formula (II) and an organic phase comprising the substituted 1,2-dihydroquinoline of Formula (I).

The process further comprises separating the aqueous phase from the organic phase. The phases may be separated using separatory funnels, industrial separators, centrifugal separators, countercurrent distributive equipment, or similar means. The aqueous phase is removed (and discarded, recycled, or regenerated) and organic phase is retained. The organic phase may be extracted with additional acid aqueous solution. The extracting and phase separating steps may be repeated two times, three times, four times, five times, six times, seven times, eight times, nine times, or more than nine times.

(iv) Isolating the Substituted 1,2-Dihydroquinoline

At the end of the process, the organic phase comprising the substituted 1,2-dihydroquinoline of Formula (I) may be subjected to evaporation to yield a concentrated preparation of the substituted 1,2-dihydroquinoline of Formula (I) or a salt thereof. Suitable evaporative means include reduced pressure evaporation, simple effect evaporation, multiple effect evaporation, or other evaporative means. The evaporation may be conducted in a rotary evaporator, a centrifugal evaporator, a natural circulation evaporator, a forced circulation evaporator, a rising film evaporator, a falling film evaporator, or a vapor-compression evaporator. The evaporation process may be conducted under a vacuum. In various embodiments, the pressure of the evaporation process may be less than about 1000 mbar, less than about 500 mbar, less than about 200 mbar, or less than about 50 mbar. In some embodiments, the evaporation may proceed at a first pressure for a period of time and then at a second pressure for another period of time. The temperature of the evaporation may proceed at a temperature from about 0° C. to about 60° C.

The amount of the substituted aniline of Formula (II) present in the purified preparation of the substituted 1,2-dihydroquinoline of Formula (I) may be determined using a quantitative method such as gas chromatography, as described above in section (I)(d). In some embodiments, the compound of Formula (I) may be crystalized (see section (II)(f) below).

(b) Extracting with Metal Sulfate Solution

Another process for removing the substituted aniline of Formula (II) from the feed stream or solution comprising the substituted 1,2-dihydroquinoline of Formula (I) and the substituted aniline of Formula (II) comprises a) contacting the feed stream/solution with a metal sulfate solution to form an aqueous phase comprising the substituted aniline of Formula (II) complexed with the metal and an organic phase comprising the substituted 1,2-dihydroquinoline of Formula (I), b) separating the organic phase from the aqueous phase, and c) isolating the substituted 1,2-dihydroquinoline of Formula (I) from the organic phase to yield of a purified preparation of the substituted 1,2-dihydroquinoline of Formula (I).

(i) Metal Sulfate Solution

A variety of metals may be used in the metal sulfate solution. Non-limiting examples of suitable metals include copper(II), nickel(II), zinc(II), silver(I), cobalt(III), or chromium(III). In one embodiment, the metal sulfate solution may comprise copper(II) sulfate dissolved in water. The copper(II) sulfate may be anhydrous or pentahydrate.

The concentration of the metal sulfate in the solution may range from about 0.3% to about 20% (w/v). In some embodiments, the concentration of metal sulfate may range from about 0.3% to about 1.0%, from about 1% to about 3%, from 3% to about 10%, or from about 10% to about 20%.

(ii) Optional Solvent

The feed stream or solution comprising the compounds of Formula (I) and Formula (II) may be used as is or it may be mixed with a suitable solvent, as described above in section (II)(a)(ii).

(iii) Phase Extraction and Isolation

The process further comprises extracting the feed stream or solution comprising the substituted 1,2-dihydroquinoline of Formula (I) and the substituted aniline of Formula (II) with the copper sulfate solution essentially as described above in section (II)(a)(iii) and then isolating the substituted 1,2-dihydroquinoline of Formula (I) from the organic phase essentially as described above in section (II)(a)(iv).

(c) Reducing the Substituted Aniline with a Nitrite

Still another process for removing the substituted aniline of Formula (II) from the feed stream or solution comprising the substituted 1,2-dihydroquinoline of Formula (I) and the substituted aniline of Formula (II) comprises contacting the feed stream/solution with a nitrite in the presence of an acid to form a derivative of the substituted aniline of Formula (II) (which is not toxic or mutagenic), and optionally removing the derivative of the substituted aniline of Formula (II) from the resultant solution.

The nitrite may be a salt comprising an alkali metal, alkaline earth metal, or ammonium ion. In certain embodiments, the nitrite may be sodium nitrite, potassium nitrite, calcium nitrite, magnesium nitrite, or ammonium nitrite. In specific embodiments, the nitrite may be sodium nitrite. The amount of nitrite contacted with the feed stream or solution comprising the compounds of Formula (I) and Formula (II) may vary. In some embodiments, the feed stream/solution may be contacted with a solution of sodium nitrite containing from about 0.05% to about 10% of sodium nitrite dissolved in 0.001-0.1 M hydrochloric acid or other suitable acid. The volume of sodium nitrite solution contacted with the feed stream/solution is essentially as described above in (II)(a)(iii). The derivative of the substituted aniline optionally may be removed from the resultant solution. Lastly, the final solution containing the substituted 1,2-dihydroquinoline of Formula (I) may be concentrated essentially as described above in section (II)(a)(iv).

(d) Treating with Ion Exchange Resin or Scavenger Resin

An alternate process for removing the substituted aniline from the feed stream or solution comprising the substituted 1,2-dihydroquinoline of Formula (I) and the substituted aniline of Formula (II) comprises contacting the feed stream/solution with an ion exchange resin or a scavenger resin to selectively remove the substituted aniline of Formula (II), thereby forming purified preparation of the substituted 1,2-dihydroquinoline of Formula (I).

Most ion exchange resins are based on crosslinked polystyrene or crosslinked acrylic or methacrylic acid polymers that are modified to contain functional groups. In some embodiments, the ion exchange resin may be a cation exchange resin, which contains carboxylic acid or sulfonic acid functional groups.

Scavenger resins may be based on crosslinked polystyrene polymers modified to contain functional groups. In certain embodiments, the scavenger resin may contain benzaldehyde, isocyanate, or isothiocyanate functional groups.

The amount of ion exchange resin or scavenger resin contacted with the feed stream or solution comprising the compounds of Formula (I) and Formula (II) can and will vary depending upon, for example, the levels of the substituted aniline of Formula (II) present in the feed stream/solution. Means for determining the appropriate amount are well-known in the art. For example, weight to weight ratio of the feed stream or solution comprising the compounds of Formula (I) and Formula (II) to the ion exchange resin or scavenger resin may range from about 1:0.001 to about 1:20. Contact between the feed stream/solution and the ion exchange or scavenger resin may be conducted using a batch process or a fixed bed (column) process. In specific embodiments, the contacting may be performed via a batch process.

Contact between the feed stream or solution comprising the compounds of Formula (I) and Formula (II) and the ion exchange resin or scavenger resin may occur at a temperature ranging from about 4° C. to about 60° C. In some embodiments, the temperature of the contacting step may range from about 0° C. to about 20° C., from about 20° C. to about 30° C., from about 30° C. to about 40° C., or from about 40° C. to about 60° C. In specific embodiments, the contacting may be performed at about room temperature.

Contact with the ion exchange resin or scavenger resin may be repeated one or more times. In some embodiments, contact between the feed stream or solution comprising the compounds of Formula (I) and Formula (II) and the ion exchange resin or scavenger resin may be repeated two times, three times, four times, five times, six times, seven times, eight times, nine times, or more than nine times.

The resultant treated solution comprising the substituted 1,2-dihydroquinoline of Formula (I) may be concentrated essentially as described above in section (II)(a)(iv).

(e) Treating with an Adsorbent

A further process for removing the substituted aniline of Formula (II) from the feed stream or solution comprising the substituted 1,2-dihydroquinoline of Formula (I) and the substituted aniline of Formula (II) comprises contacting the feed stream/solution with an adsorbent such as silica, silica gel, silica hydrogel, silicates, alumina, zeolite, bentonite, or mineral clay to adsorb the substituted aniline of Formula (II), thereby forming purified preparation of the substituted 1,2-dihydroquinoline of Formula (I).

Contact between the feed stream or solution comprising the compounds of Formula (I) and Formula (II) and the silica, silica gel, silica hydrogel, alumina, zeolite, bentonite, or mineral clay may be performed essentially as the contacting above in section (II)(d).

(f) Isolating a Salt of the Substituted 1,2-Dihydroquinoline

Another means for removing the substituted aniline of Formula (II) from the feed stream or solution comprising the substituted 1,2-dihydroquinoline of Formula (I) and the substituted aniline Formula (II) is to form a salt of the substituted 1,2-dihydroquinoline and isolate the salt by crystallization. In particular, the feed stream or solution comprising the compounds of Formula (I) and Formula (II) may be contacted with an acid (HA) to form a salt of the substituted 1,2-dihydroquinoline of Formula (Ib), as shown in the reaction scheme below:

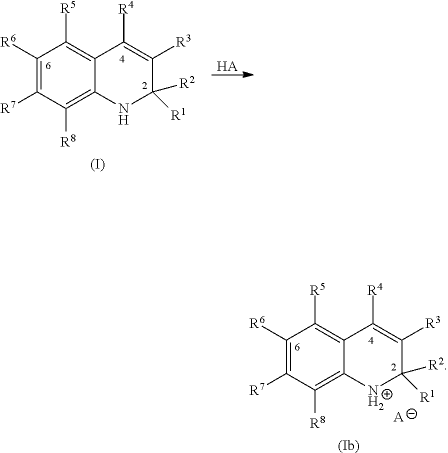

Non-limiting examples of suitable acids include acetic, algenic, anthranilic, ascorbic, aspartic, benzenesulfonic, benzoic, carbonic, citric, cyclohexylaminosulfonic, embonic (pamoic), ethanesulfonic, formic, fumaric, galactaric, galacturonic gluconic, glucuronic, glutamic, glycolic, hydrobromic, hydrochloric, hydroiodic, 4-hydroxybenzoic, hydroxybutyric, 2-hydroxyethanesulfonic, lactic, malic, maleic, mandelic, mesylic, methanesulfonic, nitric, oxalic, pantothenic, perchloric phenylacetic, phosphoric, propionic, pyruvic, salicylic, succinic, sulfanilic, sulfuric, stearic, tartaric, or toluenesulfonic acid. In specific embodiments, the acid may be hydrochloric acid, citric acid, phosphoric acid, sulfuric acid, benzoic acid, propionic acid, or formic acid.

The salt of the substituted 1,2-dihydroquinoline may be isolated by crystallization, thereby isolating the substituted 1,2-dihydroquinoline from the substituted aniline. In exemplary embodiments, the salt may be a salt of 6-ethoxy-2,2,4-trimethyl-1,2-dihydroquinoline (or ethoxyquin). For example, the salt may be ethoxyquin hydrochloride, ethoxyquin citrate, ethoxyquin phosphate, and so forth. The salt may be neutralized if necessary with the use of any base such as a metal hydroxide solution or tertiary amine to provide the free base of the substituted 1,2-dihydroquinoline

### Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The term “about,” particularly in reference to a given quantity, is meant to encompass deviations of plus or minus five percent.

The term “alkyl” as used herein describes groups containing from one to thirty carbon atoms in the principal chain. They may be straight or branched chain or cyclic and include methyl, ethyl, propyl, isopropyl, butyl, hexyl and the like.

The term “alkoxide” or “alkoxy” as used herein is the conjugate base of an alcohol. The alcohol may be straight chain, branched, cyclic, and includes aryloxy compounds.

The term “substituted” refers to moieties that are substituted with at least one atom other than carbon, including moieties in which a carbon chain atom is substituted with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thio.

### EXAMPLES

The following examples are included to demonstrate preferred embodiments of the disclosure. It should be appreciated by those of skill in the art that the techniques disclosed in the examples represent techniques discovered by the inventors to function well in the methods disclosed herein. Those of skill in the art should, however, in light of the present disclosure, appreciate that many changes can be made in the specific embodiments that are disclosed and still obtain a like or similar result without departing from the spirit and scope of the disclosure, therefore all matter set forth is to be interpreted as illustrative and not in a limiting sense.

### Example 1: Removal of PPN by Extraction with Aqueous Solution

EQ was dissolved in toluene and extracted two times with an equal volume of DI water. The toluene phase was dried over magnesium sulfate, filtered and concentrated using a rotary evaporator. Another EQ sample was dissolved in toluene and extracted two times with an equal volume of a solution of 0.1 M HCl and treated as described above. The resulting extracts were analyzed using a qualitative LC/MS method. The results indicated that the washing with DI water alone did not lower the levels of PPN. However, washing with the 0.1 M HCl solution reduced the amount of PPN in the EQ sample.

### Example 2: Optimization of Acid Extraction

To further optimize the extraction method described in Example 1, a series of HCl dilutions were used to study the effect of pH on the reduction of PPN and on the recovery of EQ. For these experiments a set of six dilute aqueous HCl solutions were prepared with pH values of 0.52, 0.7, 1.0, 2.0, 3.0, and 4.0. A 10% stock solution of EQ in toluene was prepared and 50 mL aliquots were extracted three times with the different aqueous acid solutions, respectively. The organic phase was subsequently dried, filtered and concentrated under vacuum. The concentration of PPN in the resulting samples was determined using a GC/FID method. PPN concentrations less than 10 ppm fall below the quantitation limit and are reported as BQL (below quantitation limit).

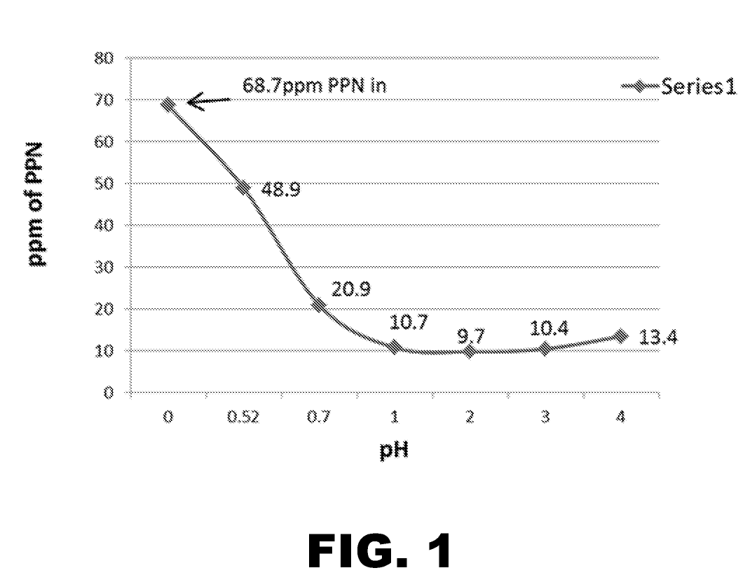

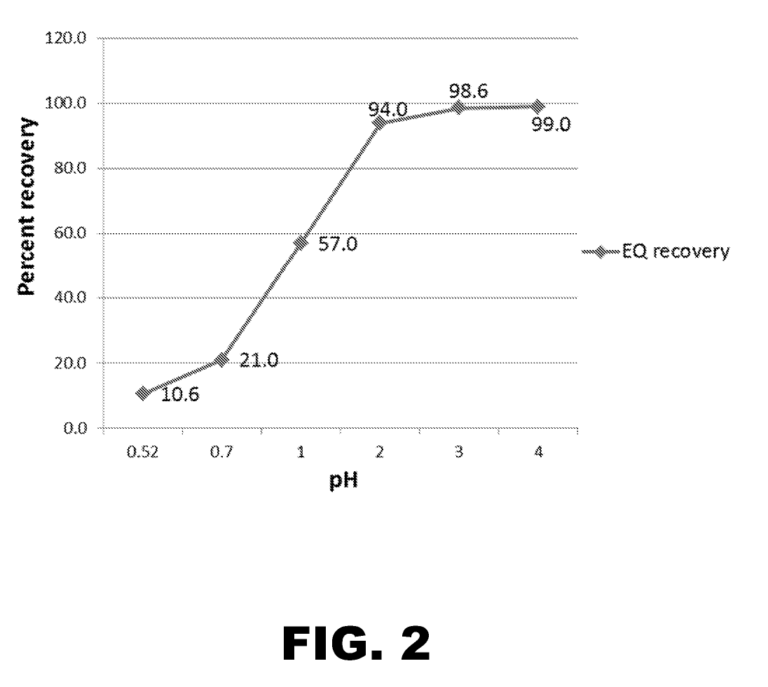

The results are presented in Table 1, FIG. 1 and FIG. 2. The levels of PPN were reduced by more than 50% when EQ was washed with an acidic solution having a pH of 0.7 or greater (FIG. 1). However, when EQ was washed with an aqueous acid solution with a pH less than 2, then the majority of the EQ was also extracted into the aqueous phase and the percent recovery is lowered (FIG. 2). Thus, it appears that the optimal pH level for aqueous acid solution is from pH 2-4.

TABLE 1
Effect of dilute acid washes on EQ recovery and PPN levels.

| | HCl | | EQ recovery | PPN in EQ |
|---|---|---|---|---|
| Sample | (M) | pH | (%) | (ppm) |
| EQ | — | — | — | 69 |
| 1 | 0.3 | 0.52 | 10.6 | 49 |
| 2 | 0.2 | 0.7 | 21.0 | 21 |
| 3 | 0.1 | 1.0 | 57.0 | 11 |
| 4 | 0.01 | 2.0 | 94.0 | BQL |
| 5 | 0.001 | 3.0 | 98.6 | BQL |
| 6 | 0.0001 | 4.0 | 99.0 | 13 |

### Example 3: Acid Accelerates PPN Formation

It was observed, however, that in some commercially available EQ samples, the levels of PPN increased over time when stored at room temperature. Addition of formic acid to these samples accelerated the increase of the PPN concentration, as shown in Table 2.

TABLE 2
PPN levels in the Absence or Presence of Added Acid.

| | Untreated | Formic Acid Treated |
|---|---|---|
| Sample | (ppm) | (ppm) |
| 1 | 18 | 40 |
| 2 | 58 | 79 |

Detailed analysis of the samples by LC/MS after addition of formic acid showed the presence of compounds with molecular weight of 257 whose concentration decreased as PPN levels increased. Thus, these unknown compounds with MW 257 were assigned structures A, B, and/or C shown below (although other isomeric imine structures are possible). It was hypothesized that these imine intermediates were formed by condensation of PPN with multiple molecules of acetone during the manufacture process. These imines may be able to reverse back to free PPN under acid conditions, such as formic acid, acting in effect as a source of PPN in those samples.

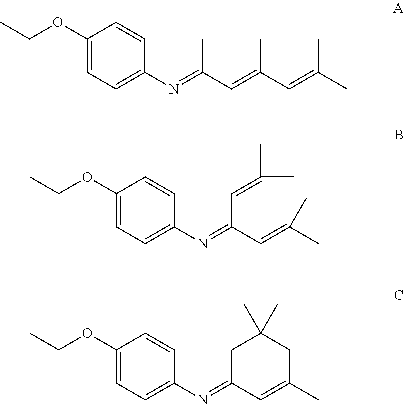

### Example 4: Preparation and Identification of Imine Byproducts

To a 100 ml round bottom flask was added 1.05 g (7.66 mmol) of PPN, 1.09 g (7.88 mmol) of isophorone and 50 ml of toluene. A catalytic amount of p-toluenesulfonic acid monohydrate was added and a stir bar. The flask was fitted with a thermocouple and a Dean Stark trap with condenser. The reaction was then heated to reflux (110° C.) for 6 hours. A small amount of water was seen in the Dean Stark trap. TLC (20% ethyl acetate/heptane) revealed a new spot between isophorone and PPN. The reaction was cooled and concentrated to dryness and dry loaded onto an 80 g silica gel column. Using a chromatograph system, the column was then eluted with 1 column volume (CV) of heptane and then a gradient from 0% to 45% of ethyl acetate for 12 CV. After approximately 8 CV, the material had eluted from the column and was collected in fraction tubes. The tubes were checked by TLC for contents and those containing only desired material were combined and concentrated to give 820 mg (3.2 mmol, 42%) of an orange oil. LC/MS (2 peaks E and Z isomers, see below) M+H 258.10.

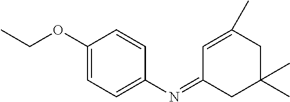 [SMILES: CCOc1ccc(/N=C2\C=C(C)CC(C)(C)C2)cc1]

### (Z)-4-ethoxy-N-(3,5,5-trimethylcyclohex-2-en-1-ylidene)aniline (structure C)

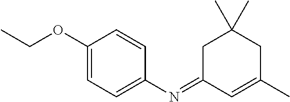 [SMILES: CCOc1ccc(/N=C2/C=C(C)CC(C)(C)C2)cc1]

### (E)-4-ethoxy-N-(3,5,5-trimethylcyclohex-2-en-1-ylidene)aniline (structure C)

(Z)-4-ethoxy-N-(3,5,5-trimethylcyclohex-2-en-1-ylidene)aniline and (E)-4-ethoxy-N-(3,5,5-trimethylcyclohex-2-en-1-ylidene)aniline were co-injected with ethoxyquin. The retention time and MW of Z)-4-ethoxy-N-(3,5,5-trimethylcyclohex-2-en-1-ylidene)aniline and (E)-4-ethoxy-N-(3,5,5-trimethylcyclohex-2-en-1-ylidene)aniline coincided with a known, but unidentified, by-product of ethoxyquin production. This by-product was then shown to revert to PPN upon treatment with formic acid. The identification of this by-product in ethoxyquin samples is important because it provides a means of identifying the impurity and may serve as a marker for potential PPN increase.

### Example 5: Removal of PPN by Silica Gel Column Chromatography

A solution of ethoxyquin (15.1 g, 68 ppm PPN) in heptane (15 mL) was added to silica gel (300 g) packed in a column and pre-wetted with heptane. The column was eluted with heptane (1CV), 0-10% ethyl acetate/heptane (1 CV) and then 10% EA/heptane (6 CV). The fractions were collected and analyzed by TLC. Fractions showing high purity were combined and the solvent was evaporated under vacuum to give EQ as a yellow oil.

The chemical stability of the EQ purified by silica gel chromatography was analyzed by heating 1 g samples in 1 dram vials at three different temperatures over the indicated period of time. The samples were then analyzed for PPN content, as shown in Table 3.

TABLE 3
Chemical Stability of Purified EQ.

| Temperature | Time | PPN (ppm) |
|---|---|---|
| RT | 7 days | BQL |
| 50° C. | 64 hrs | BQL |
| 100° C. | 64 hrs | BQL |

### Example 6: Removal of PPN by Treatment with Silica Gel

A stock solution of ethoxyquin (17.95 g, 68 ppm PPN) in heptane (122.89 g) was prepared and weighed into scintillation vials containing various amounts of silica gel (60 Å) as shown in Table 4. The resulting mixtures were stirred at room temperature for 4 hours, filtered and the filtrates were evaporated under vacuum. The resulting EQ oils were analyzed by GC/FID for PPN level.

TABLE 4
PPN Levels Following Silica Gel Treatment.

| | Silica | EQ Solution | EQ Recovery | PPN |
|---|---|---|---|---|
| Sample | (g) | (g) | (%) | (ppm) |
| 1 | 0.1041 | 10.62 | 85.0 | 35 |
| 2 | 0.2509 | 10.69 | 78.5 | 28 |
| 3 | 0.4986 | 10.63 | 76.0 | 19 |
| 4 | 1.0063 | 10.65 | 65.6 | 14 |
| 5 | 1.5083 | 10.65 | 55.3 | 11 |

### Example 7: Removal of PPN by Treatment with Alumina

A stock solution of ethoxyquin (17.95 g, 68 ppm PPN) in heptane (122.89 g) was prepared and weighed into scintillation vials containing various amounts of alumina (Type WN-6, Neutral, Activity Grade Super I) as shown in Table 5. The resulting mixtures were stirred at room temperature for 4 hours, filtered and the filtrates were evaporated under vacuum. The resulting EQ oils were analyzed by GC/FID for PPN levels, which are presented in Table 5.

TABLE 5
PPN Levels Following Alumina Treatment.

| | Alumina | EQ Solution | EQ Recovery | PPN |
|---|---|---|---|---|
| Sample | (g) | (g) | (%) | (ppm) |
| 1 | 0.1032 | 10.63 | 92.3 | 38 |
| 2 | 0.2537 | 10.67 | 85.3 | 27 |
| 3 | 0.5034 | 10.66 | 88.3 | 18 |
| 4 | 1.0002 | 10.67 | 84.6 | 12 |
| 5 | 1.5063 | 10.65 | 79.6 | BQL |

### Example 8: Removal of PPN by Extraction with Copper(II) Sulfate Solution

A solution of ethoxyquin (11.22 g, 68 ppm PPN) in heptane (78.31 g), was prepared. The stock solution (˜50 mL) was loaded into a separatory funnel and extracted with a 10% copper(II) sulfate solution (3×50 mL) and DI water (1×50 mL). The organic solution was then dried over magnesium sulfate, filtered and evaporated under vacuum to give EQ as an oil, which was analyzed for PPN. The level of PPN was BQL, with a 95.5% recovery of EQ.

### Example 9: Removal of PPN by Treatment with Silica Hydrogel

A stock solution of ethoxyquin (9.23 g; 68 ppm PPN) in heptane (61.47 g) was prepared. The stock solution was weighed into scintillation vials containing varying amounts of silica hydrogel (Sorbsil R92) as shown in Table 6. The resulting mixtures were stirred at room temperature. After 4 hours the mixtures were filtered and the filtrates were evaporated under vacuum. The levels of PPN in the resulting EQ oils are presented in Table 6.

TABLE 6
PPN Levels Following Silica Hydrogel Treatment.

| | Silica | EQ Solution | EQ Recovery | PPN |
|---|---|---|---|---|
| Sample | (g) | (g) | (%) | (ppm) |
| 1 | 0.109 | 10.72 | 88.6 | 12 |
| 2 | 0.202 | 10.70 | 88.1 | 12 |
| 3 | 0.515 | 10.64 | 85.7 | 11 |
| 4 | 1.03 | 10.71 | 79.4 | BQL |
| 5 | 1.526 | 10.75 | 72.7 | BQL |

### Example 10: Removal of PPN by Treatment with Basic or Acidic Alumina

A stock solution of ethoxyquin (10.91 g, 68 ppm PPN) in heptane (73.47 g) was prepared. The stock solution was weighed into scintillation vials containing varying amounts of Brockman I acidic alumina (A) or Brockman I basic alumina (B) as shown in Table 7 below. The resulting mixtures were stirred at room temperature for 6 hours and then filtered. The filtrates were evaporated under vacuum. The levels of PPN in the resulting oils are shown in Table 7.

TABLE 7
PPN Levels Following Alumina Treatment.

| | Alumina | Alumina | EQ Solution | EQ Recovery | PPN |
|---|---|---|---|---|---|
| Sample | Type | (g) | (g) | (%) | (ppm) |
| 1 | A | 0.515 | 10.66 | 87.8 | BQL |
| 2 | A | 1.034 | 10.69 | 83.9 | BQL |
| 3 | A | 1.502 | 10.65 | 79.2 | BQL |
| 4 | B | 0.495 | 10.70 | 87.5 | BQL |
| 5 | B | 1.007 | 10.72 | 83.0 | BQL |

### Example 11: Removal of PPN by Extraction with Sodium Nitrite Solution

Two solutions of ethoxyquin were prepared by weighing ethoxyquin (68 ppm PPN) and (A=5.23 g EQ and 33.74 g heptane; B=5.27 g EQ and 34.47 g heptane). Each solution was loaded into a separatory funnel and extracted with a sodium nitrite solution (˜200 mg of sodium nitrite dissolved into 50 mL of an HCl solution), and then with 50 mL of DI water. The organic solution was then dried over magnesium sulfate, filtered and evaporated under vacuum. The levels of PPN in the resulting EQ oils are presented in Table 8.

TABLE 8
PPN Levels Following Sodium Nitrite Treatment.

| | EQ Solution | NaNO₂ | HCl | EQ Recovery | PPN |
|---|---|---|---|---|---|
| Sample | (g) | (g) | solution | (%) | (ppm) |
| A | 38.97 | 0.197 | 0.01M | 97.1 | 10 |
| | | | (pH = 2.20) | | |
| B | 39.74 | 0.202 | 0.001M | 97.7 | 18 |
| | | | (pH = 3.08) | | |

### Example 12: Removal of PPN by Treatment with Benzaldehyde Resin

Four scintillation vials were loaded with a benzaldehyde resin (4-Benzyloxybenzaldehyde, polymer-supported), ethoxyquin (68 ppm PPN), dichloromethane, and acetic acid as noted in Table 9. The mixtures were stirred overnight, filtered, and solvents were evaporated under vacuum. The levels of PPN in the resulting EQ oils are shown in Table 9.

TABLE 9
PPN Levels Following Benzaldehyde Resin Treatment

| | | | | | EQ | |
|---|---|---|---|---|---|---|
| | PS-CHO | EQ | DCM | HOAc | Recovery | PPN |
| Sample | (g) | (g) | (g) | (μL) | (%) | (ppm) |
| 1 | 0.0313 | 1.554 | 19.587 | 0.00 | 96.5 | 23 |
| 2 | 0.0312 | 1.445 | 19.008 | 10 | 98.3 | 20 |
| 3 | 0.1027 | 1.476 | 19.116 | 0.00 | 96.9 | 18 |
| 4 | 0.1003 | 1.449 | 18.967 | 10 | 96.6 | 17 |

### Example 13: Removal of PPN by Treatment with Isocyanate Resin

Four scintillation vials were loaded with isocyanate resin (STRATOSPHERES™ PL-NCO resin 150-300 μm, extent of labeling: 1.5 mmol/g loading, 1% cross-linked), ethoxyquin (68 ppm PPN), and dichloromethane as noted in Table 10. The mixtures were stirred overnight (˜20 hours), filtered and the filtrates were evaporated under vacuum. The levels of PPN in the resulting EQ oils are shown in Table 10.

TABLE 10
PPN Levels Following Isocyanate Resin Treatment.

| | | | | Isolated | EQ | |
|---|---|---|---|---|---|---|
| | PS-NCO | EQ | DCM | EQ | Recovery | PPN |
| Sample | (g) | (g) | (g) | (g) | (%) | (ppm) |
| 1 | 0.0357 | 1.69 | 19.33 | 1.68 | 99.4 | 26 |
| 2 | 0.1062 | 1.62 | 19.65 | 1.62 | 99.9 | 20 |
| 3 | 0.2156 | 1.59 | 19.67 | 1.55 | 97.5 | 15 |

### Example 14: Removal of PPN by Treatment with Aluminosilicate Mineral Clay

A stock solution of ethoxyquin (8.36 g, 68 ppm PPN) in heptane (55.83 g) was weighed into scintillation vials containing varying amounts of aluminosilicate mineral clay (SOLIS®, Novus International) as shown in Table 11. The resulting mixtures were stirred at room temperature for 4 hours, filtered and the filtrates were evaporated under vacuum. The levels of PPN in the resulting EQ oils are presented in Table 11.

TABLE 11
PPN Levels Following Aluminosilicate Mineral Clay Treatment.

| | SOLIS ® | EQ | EQ Recovery | PPN |
|---|---|---|---|---|
| Sample | (g) | (g) | (%) | (ppm) |
| 1 | 0.0996 | (~15 mL) | 90.7 | 18.5 |
| 2 | 0.2525 | 10.76 | 89.9 | 14.2 |
| 3 | 0.503 | 10.73 | 88.0 | 10.1 |
| 4 | 1.004 | 10.70 | 83.2 | BQL |
| 5 | 1.514 | 10.74 | 77.2 | BQL |

### Example 15: Removal of PPN by Multiple Treatments with Aluminosilicate Mineral Clay at Ambient Temperature

An ethoxyquin (20.06 g, 51.1 ppm PPN) and aluminosilicate mineral clay (2.01 g, SOLIS®, Novus International) mixture was stirred at room temperature for 1 hour and then filtered through a fritted tube into a round bottom flask to give 17.42 g (87%) of an oil with 41.9 ppm PPN by GC/FID. To the resulting ethoxyquin (13.97 g, 41.9 ppm PPN) was added aluminosilicate mineral clay (1.43, SOLIS®, Novus International) and the mixture was stirred at room temperature for 1 hour and then filtered through a fritted tube into a round bottom flask to give 12.05 g (86%) of an oil with 33.3 ppm PPN by GC/FID. To the resulting ethoxyquin (8.65 g, 33.3 ppm PPN) was added aluminosilicate mineral clay (1.77 g, SOLIS®, Novus International) and the mixture was stirred at room temperature for 1 hour and then filtered through a fritted tube into a round bottom flask to give 5.79 g (67%) of an oil with 24 ppm of PPN by GC/FID.

### Example 16: Removal of PPN by Multiple Treatments with Aluminosilicate Mineral Clay at Elevated Temperature

An ethoxyquin (20.06 g, 61.2 ppm PPN) and aluminosilicate mineral clay (2.00 g, SOLIS®, Novus International) mixture was stirred at 40° C. for 1 hour and then filtered through a fritted tube containing celite (0.44 g) into a round bottom flask to give 16.88 g (84%) of an oil with 46.7 ppm PPN by GC/FID. To the resulting ethoxyquin (13.55 g, 46.7 ppm PPN) was added aluminosilicate mineral clay (1.41 g, SOLIS®, Novus International) and the mixture was stirred at 40° C. for 1 hour and then filtered through a fritted tube into a round bottom flask to give 11.27 g (83%) of an oil with 32.3 ppm PPN by GC/FID. To the resulting ethoxyquin (7.91 g, 32.3 ppm PPN) was added aluminosilicate mineral clay (1.58 g, SOLIS®, Novus International) and the mixture was stirred at room temperature for 1 hour and then filtered through a fritted tube into a round bottom flask to give 6.17 g (78%) of an oil with 14.2 ppm of PPN by GC/FID.

### Example 17: Removal of PPN by an Aluminosilicate Column

A solution of ethoxyquin (10 g, 48.9 ppm PPN) in hexane (34.2 g) was eluted through a 25 mL SPE tube containing aluminosilicate mineral clay (4 g, SOLIS®, Novus International), pre-wet with hexane, using positive pressure of nitrogen over approximately 10 min. The solvent was evaporated at 40° C./20 mbar for 1 hr to give 9.37 g (93.6%) of an oil containing 9.3 ppm of PPN by GC/FID.

### Example 18: Removal of PPN by Treatment with Formic Acid

A solution of ethoxyquin (29.8 g, 41.6 ppm PPN) in 90.3 g of hexanes was treated with 30 mL of a 13% formic acid solution for two hours with stirring, under nitrogen. After this time, the material was poured into a separatory funnel and the layers separated. The organic layer was washed with 40 mL of 0.1N NaOH, followed by 40 mL of DI water. The pH of the DI water was neutral after the wash. The organic layer was then concentrated at 40° C./185 mbar for 15 min then at 40° C./32 mbar for 1.5 hours to give 25.3 g (84.9%) of an orange oil containing 30.7 ppm of PPN.

### Example 19: Removal of PPN by Treatment Twice with Formic Acid

A solution of ethoxyquin (20.1 g; 41.6 ppm PPN) in 60.0 g of hexanes was treated with 45 mL of a 13% formic acid solution for 1.5 hours with stirring, under nitrogen. After this time, the material was poured into a separatory funnel and the layers separated. The organic layer was treated a second time with 45 mL of a 13% formic acid solution for 1.5 hours with stirring, under nitrogen. The material was poured into a separatory funnel and the layers separated. The organic layer was washed with 100 mL of 0.1N NaOH, followed by 100 mL of DI water. The pH of the DI water was neutral after the wash. The organic layer was concentrated at 40° C./185 mbar for 15 min then at 40° C./32 mbar for 1.5 hours to give 11.8 g (59.0%) of an orange oil containing 16.1 ppm of PPN.

### Example 20: Removal of PPN by Treatment Three Times with Formic Acid

A solution of ethoxyquin (20.1 g; 41.6 ppm PPN) dissolved in 60.0 g of hexanes was treated with 45 mL of a 13% formic acid solution for 1.5 hours with stirring, under nitrogen. The material was poured into a separatory funnel and the layers separated. The organic layer was treated a second time with 45 mL of 13% formic acid solution for 1.5 hours with stirring, under nitrogen. The material was poured into a separatory funnel and the layers separated. The organic layer was treated a third time with 45 mL of 13% formic acid solution for 1.5 hours with stirring, under nitrogen. The material was poured into a separatory funnel and the layers separated. The organic layer was washed with 100 mL of 0.1N NaOH, followed by 100 mL of DI water. The pH of the DI water was neutral after the wash. The organic layer was concentrated at 40° C./185 mbar for 15 min then at 40° C./32 mbar for 1.5 hours to give 8.7 g (43.1%) of an orange oil containing 12.7 ppm of PPN.

### Example 21: Removal of PPN by Multiple Treatments with Alumina

A solution of ethoxyquin (10.02 g; 41.6 ppm PPN) in 32.3 g of hexanes was treated with 1.0 g of alumina (acidic, Brockman I) for 1 hour with stirring under nitrogen. The alumina was filtered off and the filtrate was treated with 1.00 g of alumina (acidic, Brockman I) and stirred for 1 hour under nitrogen. The alumina was filtered and the filtrate treated with 1.96 g of alumina (acidic Brockman I) and stirred for 1 hour under nitrogen. The alumina was filtered and the filtrate was concentrated at 40° C./185 mbar for 15 min then at 40° C./30 mbar for one hour to give 8.56 g (85.4%) of an orange oil containing 10.3 ppm of PPN.

### Example 22: Removal of PPN by Multiple Treatments with Alumina at Ambient Temperature

An ethoxyquin (20.21 g, 51.1 ppm PPN) and alumina (2.01 g, acidic, Brockman I) mixture was stirred at room temperature for 1 hour and then filtered through a fritted tube into a round bottom flask to give 17.40 g (86%) of an oil with 18.4 ppm PPN by GC/FID. To the resulting ethoxyquin (13.15 g, 18.4 ppm PPN) was added alumina (1.46 g, acidic, Brockman I) and the mixture was stirred at room temperature for 1 hour and then filtered through a fritted tube into a round bottom flask to give 11.89 g (90%) of an oil with 12.9 ppm PPN by GC/FID. To the resulting ethoxyquin (8.59 g, 12.9 ppm PPN) was added alumina (1.70 g, acidic, Brockman I) and the mixture was stirred at room temperature for 1 hour and then filtered through a fritted tube into a round bottom flask to give 5.55 g (65%) of an oil with BQL of PPN by GC/FID.

### Example 23: Removal of PPN by Multiple Treatments with Alumina at Elevated Temperature

An ethoxyquin (20.02 g, 61.2 ppm PPN) solution was mixed with alumina (2.00 g, acidic, Brockman I) by stirring at 40° C. for 1 hour and then filtered through a fritted tube into a round bottom flask to give 17.51 g (87%) of an oil with 28.8 ppm PPN by GC/FID. To the resulting ethoxyquin (14.17 g, 28.8 ppm PPN) was added alumina (1.40 g, acidic, Brockman I) and the mixture was stirred at 40° C. for 1 hour and then filtered through a fritted tube into a round bottom flask to give 11.93 g (84%) of an oil with 14.6 ppm PPN by GC/FID. To the resulting ethoxyquin (8.71 g, 14.6 ppm PPN) was added alumina (1.70 g, acidic, Brockman I) and the mixture was stirred at room temperature for 1 hour and then filtered through a fritted tube into a round bottom flask to give 6.41 g (74%) of an oil with BLQ of PPN by GC/FID.

### Example 24: Removal of PPN by an Alumina Column

A solution of ethoxyquin (10 g, 48.9 ppm PPN) in hexane (34.4 g) was eluted through a 25 mL SPE tube of alumina (4 g, acidic, Brockman I, pre-wet with hexane) over approximately 10 min. The solvent was removed from the eluent at 40° C./20 mbar for 1 hr to give 9.09 g (90.5%) of an oil containing BQL of PPN by GC/FID.

### Example 25: Removal of PPN by Treatment with Formic Acid and Treatment with Alumina

Ethoxyquin (10.4 g) from Example 18 was dissolved in 50 mL of hexanes and treated with 1.0 g of alumina (acidic, Brockman I) for 1 hour with stirring under nitrogen. The alumina was filtered and the filtrate was treated with 0.99 g of alumina (acidic, Brockman I) and stirred for 1 hour under nitrogen. The alumina was filtered and the filtrate treated with 1.99 g of alumina (acidic, Brockman I) and stirred for 1 hour under nitrogen. The alumina was filtered and the filtrate was at 40° C./185 mbar for 15 min then at 40° C./30 mbar for one hour to give 8.80 g (84.8%) of an orange oil containing BQL of PPN.

### Example 26: Removal of PPN by Treatment with Formic Acid and Treatment with Silica

Ethoxyquin (10.0 g) from Example 18 was dissolved in 50 mL of hexanes and treated with 0.98 g of silica (Davisil Grade 634, 100-200 mesh) for 1 hour with stirring under nitrogen. The silica was filtered and the filtrate was treated with 0.97 g of silica (Davisil Grade 634, 100-200 mesh) and stirred for 1 hour under nitrogen. The silica was filtered and the filtrate treated with 1.96 g of silica (Davisil Grade 634, 100-200 mesh) and stirred for 1 hour under nitrogen. The silica was filtered and the filtrate was concentrated at 40° C./185 mbar for 15 min then at 40° C./30 mbar for one hour to give 7.19 g (72.2%) of an orange oil containing 25.4 ppm of PPN.

### Example 27: Removal of PPN by Ethoxyquin Salt Preparation

Ethoxyquin (2 g, 68 ppm PPN) was dissolved in 4:1 (v/v) acetone-water solution (30 mL) and treated with activated carbon (1.5 g) with stirring. After a certain amount of time, concentrated hydrochloric acid (1 mL) was added to the stirring solution and the reaction was heated to 55° C. open to air for 1 hour. The hot solution was then filtered and concentrated under reduced pressure (to about 4 mL total solution). Acetone (50 mL) was then added and the solution was left at 5° C. to crystallize. The resulting crystals were collected by filtration and washed with toluene to give 1.1 g (56%) of ethoxyquin hydrochloride as white crystals containing BQL of PPN.

What is claimed is:

1. A composition comprising a compound of Formula (I) or salt thereof and less than 40 ppm of a compound of Formula (II), wherein the compound of Formula (I) is produced in a batch of at least one metric ton by reacting the compound of Formula (II) with a compound comprising a carbonyl group, the compounds of Formulas (I) and (II): 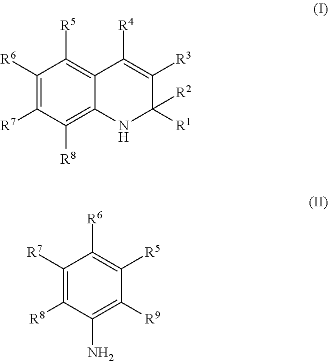
wherein:
R¹, R², R³ and R⁴ are independently hydrogen, C₁-C₆ alkyl, or C₁-C₆ substituted alkyl;
R⁵, R⁷, and R⁹ are independently hydrogen, C₁-C₆ alkyl, or C₁-C₆ substituted alkyl; and
R⁶ and R⁸ are independently hydrogen, C₁-C₆ alkoxy, or C₁-C₆ substituted alkoxy group, provided that one is other than hydrogen.

2. The composition of claim 1, wherein R¹, R², R³ and R⁴ are independently hydrogen or methyl; R⁵, R⁷, and R⁹ are hydrogen, and R⁶ and R⁸ are independently hydrogen or ethoxy.

3. The composition of claim 2, wherein each of R¹, R², and R⁴ is methyl, R³ and R⁸ are hydrogen, and R⁶ is ethoxy.

4. The composition of claim 1, wherein the composition comprises less than about 20 ppm of the compound of Formula (II).

5. The composition of claim 4, wherein the composition comprises less that about 10 ppm of the compound of Formula (II).

6. The composition of claim 1, wherein the composition is substantially devoid of water and/or solvent.

7. The composition of claim 1, wherein the level of the compound of Formula (II) is stable for at least three months when the composition is stored at room temperature; wherein the level of the compound of Formula (II) is stable in the presence of an acid; and/or the composition is substantially devoid of imine derivatives of the compound of Formula (I).

8. A process for removing a compound of Formula (II) from a feed stream comprising a compound of Formula (I) and the compound of Formula (II), the process comprising:
(a) contacting the feed stream comprising the compounds of Formula (I) and Formula (II) with an aqueous solution having a pH from about 2 to 4 to form an aqueous phase comprising the compound of Formula (II) and an organic phase comprising the compound of Formula (I);
(b) separating the phases; and
(c) isolating the compound of Formula (I) from the organic phase to yield a purified preparation of the compound of Formula (I), wherein the purified preparation of the compound of Formula (I) contains less than about 70 ppm of the compound of Formula (II);
the compounds of Formulas (I) and (II): 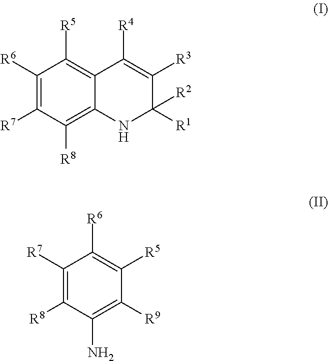
wherein:
R¹, R², R³ and R⁴ are independently hydrogen, C₁-C₆ alkyl, or C₁-C₆ substituted alkyl;
R⁵, R⁷, and R⁹ are independently hydrogen, C₁-C₆ alkyl, or C₁-C₆ substituted alkyl; and
R⁶ and R⁸ are independently hydrogen, C₁-C₆ alkoxy, or C₁-C₆ substituted alkoxy group, provided that one is other than hydrogen.

9. The process of claim 8, wherein R¹, R², R³ and R⁴ are independently hydrogen or methyl; R⁵, R⁷, and R⁹ are hydrogen, and R⁶ and R⁸ are independently hydrogen or ethoxy.

10. The process of claim 9, wherein each of R¹, R², and R⁴ is methyl, R³ and R⁸ are hydrogen, and R⁶ is ethoxy.

11. The process of claim 8, wherein the purified preparation of the compound of Formula (I) contains less than 40 ppm of the compound of Formula (II).

12. The process of claim 11, wherein the purified preparation of the compound of Formula (I) contains less than 20 ppm of the compound of Formula (II).

13. The process of claim 12, wherein the purified preparation of the compound of Formula (I) contains less than 10 ppm of the compound of Formula (II).

14. The process of claim 8, wherein the aqueous solution having a pH from about 2 to 4 is chosen from a solution comprising hydrochloric acid, nitric acid, phosphoric acid, sulfuric acid, formic acid, acetic acid, or a metal sulfate.

15. The process of claim 8, wherein a nonpolar solvent is mixed with the feed stream comprising the compounds of Formula (I) and Formula (II) before contact with the aqueous solution having a pH from about 2 to 4.

16. The process of claim 8, wherein steps (a) and (b) are repeated up to four times.

17. The process of claim 8, wherein step (c) comprises crystallizing a salt of the compound of Formula (I).

18. The process of claim 8, wherein the process further comprises contacting the purified preparation of the compound of Formula (I) with a resin.

19. The process of claim 18, wherein the resin is an ion exchange resin, a scavenger resin, or an adsorbent.

20. The process of claim 18, wherein contacting with the resin is repeated up to four times.

21. A process for removing a compound of Formula (II) from a feed stream comprising a compound of Formula (I) and the compound of Formula (II), the process comprising contacting the feed stream comprising the compounds of Formula (I) and Formula (II) with a resin to selectively remove the compound of Formula (II) and yield a purified preparation of the compound of Formula (I), wherein the purified preparation of the compound of Formula (I) contains less than about 70 ppm of the compound of Formula (II);
the compounds of Formulas (I) and (II): 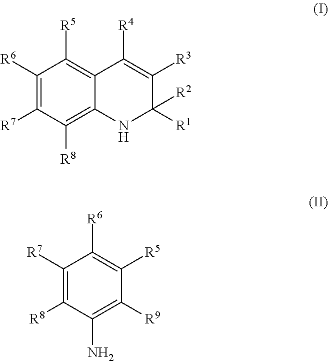
wherein:
R¹, R², R³ and R⁴ are independently hydrogen, C₁-C₆ alkyl, or C₁-C₆ substituted alkyl;
R⁵, R⁷, and R⁹ are independently hydrogen, C₁-C₆ alkyl, or C₁-C₆ substituted alkyl; and
R⁶ and R⁸ are independently hydrogen, C₁-C₆ alkoxy, or C₁-C₆ substituted alkoxy group, provided that one is other than hydrogen.

22. The process of claim 21, wherein R¹, R², R³ and R⁴ are independently hydrogen or methyl; R⁵, R⁷, and R⁹ are hydrogen, and R⁶ and R⁸ are independently hydrogen or ethoxy.

23. The process of claim 22, wherein each of R¹, R², and R⁴ is methyl, R³ and R⁸ are hydrogen, and R⁶ is ethoxy.

24. The process of claim 21, wherein the purified preparation of the compound of Formula (I) contains less than 40 ppm of the compound of Formula (II).

25. The process of claim 24, wherein the purified preparation of the compound of Formula (I) contains less than 20 ppm of the compound of Formula (II).

26. The process of claim 25, wherein the purified preparation of the compound of Formula (I) contains less than 10 ppm of the compound of Formula (II).

27. The process of claim 21, wherein the resin is an ion exchange resin, a scavenger resin, or an adsorbent.

28. The process of claim 27, wherein the adsorbent is alumina, silica, silica gel, silica hydrogel, zeolite, bentonite, or mineral clay.

29. The process of claim 21, wherein contacting with the resin is repeated up to four times.

30. The process of claim 21, wherein the process further comprises isolating the compound of Formula (I) from the purified preparation of the compound of Formula (I).

31. The process of claim 30, wherein the isolating comprises forming a salt of the compound of Formula (I) and crystallizing the salt of the compound of Formula (I).
